(19) United States
(12) Patent Application Publication
(10) Pub. No.: US 2006/0105433 A1
(43) Pub. Date: May 18, 2006

(54) Rapid thermocycler

(76) Inventors: William D. Bickmore JR., Saint George, UT (US); Gilbert M. Jennings, St. George, UT (US)

Correspondence Address: HOLME ROBERTS & OWEN, LLP, Suite 1800, 299 South Main, Salt Lake City, UT, 84111, US

(21) Appl. No.: 10/991,746

(22) Filed: Nov. 18, 2004

(51) Int. Cl. C12M 1/38; C12P 19/34

(52) U.S. Cl. 435/91.2; 435/286.5; 435/303.1; 435/809; 165/206

(57) ABSTRACT

A thermocycler is provided, that in one embodiment has separate heat exchangers for each thermocycler target temperature, and a cold boost heat exchanger and a hot boost heat exchanger. Fluid conduit is used to circulate fluid through the appropriate heat exchanger and through a sample holder containing at least one sample. Each heat exchanger has sufficiently high thermal mass to be susceptible to being adjusted to and maintained at a constant temperature, but the remaining components of the heat exchanger are preferably of low thermal mass so as to improve efficiency of the system. A small volume of circulating fluid is preferably used. In use, the temperature of the samples is increased or decreased rapidly by first passing the circulating fluid to the hot boost or cold boost heat exchanger, followed by passing the circulating fluid to the appropriate target temperature heat exchanger. A controller is used to control the heating, cooling, and duration of the various aspects of a thermocycler cycle.

### CROSS-REFERENCE TO RELATED APPLICATIONS

Not applicable.

### BACKGROUND OF THE INVENTION

1. The Field of the Invention

The present invention is directed to thermocyclers.

2. The Relevant Technology

A number of industrial, technology and research applications utilize thermal cycling to manage applications such as chemical or biochemical reactions or analytical applications.

One important tool in the field of molecular biology which utilizes thermal cycling is the process known as “polymerase chain reaction” (PCR). PCR generates large quantities of genetic material from small samples of the genetic material. This is important because small samples of genetic material may be difficult or expensive to measure or analyze or use for any practical purpose, whereas the ability to produce large amounts of desired genetic material through the PCR amplification process allows one to engage in important actions such as the identification of particular genetic material in a sample, or the measurement of how much genetic material was present, or generation of enough genetic material for use to serve as a component of further applications.

The PCR process is performed in a small reaction vial containing components for DNA duplication: the DNA to be duplicated, the four nucleotides which are assembled to form DNA, two different types of synthetic DNA called “primers” (one for each of the complementary strands of DNA), and an enzyme called DNA polymerase.

DNA is double stranded. The PCR process begins by separating the two strands of DNA into individual complementary strands, a step which is generally referred to as “denaturation.” This is typically accomplished by heating the PCR reaction mixture to a temperature of about 94 to about 96 degrees centigrade for a period of time between a few seconds to over a minute in duration.

Once the DNA is separated into single strands, the mixture is cooled to about 45 to about 60 degrees centigrade (typically chosen to be about 5 degrees below the temperature at which the primer will melt) in order to allow a primer to bind to each of the corresponding single strands of DNA in the mixture. This step is typically called “annealing.” The annealing step typically takes anywhere from a few seconds up to a few minutes.

Next, the reaction vessel is heated to about 72 to 73 degrees centigrade, a temperature at which DNA polymerase in the reaction mixture acts to build a second strand of DNA onto the single strand by adding nucleic acids onto the primer so as to form a double stranded DNA that is identical to that of the original strand of DNA. This step is generally called “extension.” The extension step generally takes from a few seconds to a couple minutes to complete.

This series of three steps, also sometimes referred to as “stages”, define one “cycle.” Completion of a PCR cycle results in doubling the amount of DNA in the reaction vial. Repeating a cycle results in another doubling of the amount of DNA in the reaction vial. Typically, the process is repeated many times, e.g. 10 to 40 times, resulting in a large number of identical pieces of DNA. Performing 20 cycles results in more than a million copies of the original DNA sample. Performing 30 cycles results in more than a billion copies of the original DNA sample. A “thermocycler” is used to automate the process of moving the reaction vessel between the desired temperatures for the desired period of time.

It generally takes about three hours to run about 30 cycles when using conventional equipment. This amount of time is required because of the time that is spent accomplishing a change of temperature between each PCR step, as well as the time required at each target temperature. Although the ability to make over a million copies in only three hours was a tremendously important advance in the field of molecular biology, it would be of great value to be able to decrease the time required to run each cycle. U.S. Pat. No. 6,787,338 and US Publication No. 2004/0086927, the disclosures of which are incorporated herein by reference, both offer proposed solutions to the problems associated with attempting to perform PCR cycles more rapidly.

### BRIEF SUMMARY OF THE INVENTION

The present invention provides a thermocycler useful for performing PCR or other applications requiring thermal cycling.

An embodiment of the thermocycler has separate heat exchangers for each thermocycler target temperature, and a cold boost heat exchanger and a hot boost heat exchanger.

Fluid conduit is used to circulate fluid through the appropriate heat exchanger and through a sample holder containing at least one sample. Each heat exchanger has sufficiently high thermal mass to be susceptible to being adjusted to and maintained at a constant temperature, but the remaining components of the heat exchanger are preferably of low thermal mass so as to improve efficiency of the system. A small volume of circulating fluid is preferably used.

In use, the temperature of the samples is increased or decreased rapidly by first passing the circulating fluid to the hot boost or cold boost heat exchanger, followed by passing the circulating fluid to the appropriate target temperature heat exchanger.

A controller is used to control the heating, cooling, and duration of the various aspects of a thermocycler cycle.

These and other objects and features of the present invention will become more fully apparent from the following description and appended claims, may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates in schematic form an embodiment of a thermocycler in accordance with the present invention.

FIG. 2 shows schematically aspects of a reaction vial holder of FIG. 1.

FIG. 3 shows additional detail with respect to the reaction vial holder of FIG. 2.

FIG. 4 is a graph illustrating the temperature over time of a reaction mixture in a conventional PCR thermocycler.

FIG. 5 is a graph showing how the rate of temperature increase is reduced as a target temperature is approached in a conventional PCR thermocycler.

FIG. 6 is a graph illustrating overshoot in some conventional PCR thermocyclers.

FIG. 7 is a graph illustrating ringing in some conventional PCR thermocyclers.

FIG. 8 is a graph showing the temperature of circulating fluid in the thermocycler of FIG. 1.

FIG. 9 is a graph showing a typical cycle of the thermocycler of FIG. 1.

### DETAILED DESCRIPTION OF THE PREFERRED EMBODIMENTS

The present invention provides a rapid thermocycler useful for performing PCR or other applications requiring thermal cycling. For purposes of illustrating the concepts of the invention, the following description shall describe the thermocycler of the present invention in the context of the PCR process, but one of ordinary skill will appreciate from the teachings herein how it can be applied to other uses.

Conventional thermocyclers have taken a number of forms. Perhaps the most common structure incorporates a large, solid, thermally conductive block having wells formed therein adapted to receive small reaction vessels. In the context of a thermocycler for use with performing PCR, a conventional block contains a number of conical-like wells, typically 96 wells, that accept reaction vials of a corresponding size and shape. A large metal block is used to provide a large thermal mass that is intended to bring all of the reaction vials to the correct reaction temperature simultaneously, and to hold them at the same temperature throughout the intended reaction duration. This is important so that one can insure that every vial proceed to a similar degree along the reaction path during the course of a cycle of the thermocycler. Failure to maintain all of the reaction vials at the appropriate temperature can, for example, result in a failure in one or more vials to properly denature, anneal or extend the contents of affected vials.

Although many thermocyclers utilize large blocks of this sort, such large blocks cause problems of their own. One of those problems is the significant amount of time required to cycle the temperature of the large block to each target temperature.

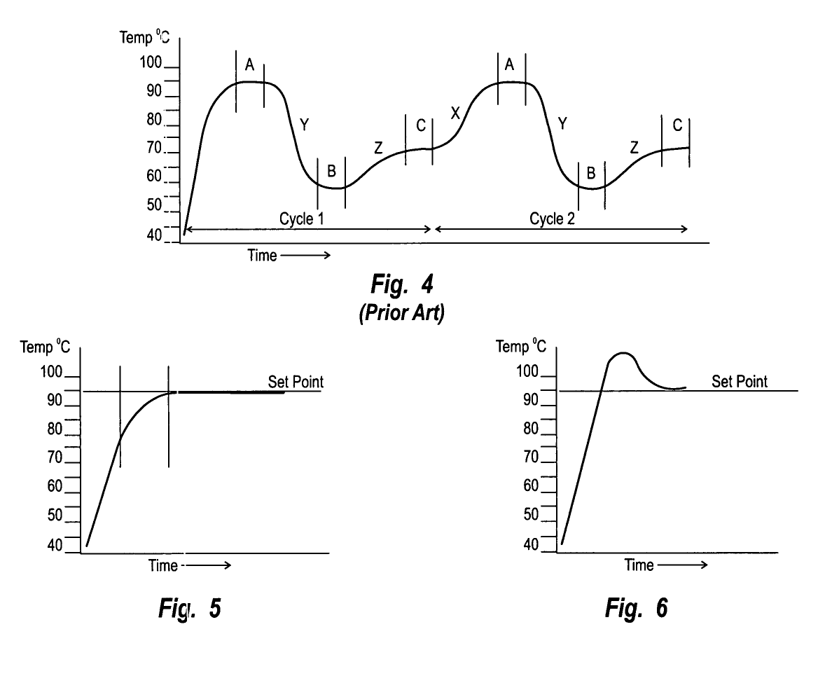

In an ideal system, the temperature would be changed instantly between each of the PCR steps, because that would result in the most rapid and most predictable amplification. Such an ideal system would look like a square wave. In actuality, a significant amount of time is required within which to change the temperature of the large blocks of common thermocyclers, as may be appreciated by reference to FIG. 4, which depicts the first two cycles of a conventional thermocycler that utilizes a large thermal mass block of the type described above. In FIG. 4, region A depicts a constant temperature that is maintained for sufficient time to effect denaturation of a DNA sample. Curve X depicts the heating of the block to the proper temperature for denaturation. Curve X illustrates there is a significant time lag from the moment that a temperature change is initiated until the block finally reaches the desired temperature.

Regions B and C, respectively, depict the cooling and heating of the large block so as to effect annealing and extension. Curve Y depicts the decrease in temperature of the block over time leading to annealing, and curve Z depicts the increase of temperature over time leading to the extension step. FIG. 4 depicts two cycles of the thermocycler; more cycles may follow.

FIG. 4 illustrates that a substantial amount of the total time required to perform each cycle is utilized to move the temperature of the large block from one desired temperature to the next. This problem is exacerbated by the difficulty of halting an increase or decrease in temperature immediately; unless the rate of temperature change is slowed as the target temperature is approached, the block temperature will overshoot the target temperature.

FIG. 5 illustrates the increase in a temperature of the large block of a conventional thermocycler, which is initially a steep curve, but then enters a region where the rate of temperature increase is slowed so as to prevent overshoot of the target temperature. This need to reduce the rate of temperature change near the target temperature not only prolongs the time, but also results in elongation of the period of time where the block is near the target temperature, which can affect the contents of the reaction vial.

FIG. 6 depicts the approach taken in some systems to avoid the delay illustrated in FIG. 5, which involves an intentional or unintentional “overshoot.” This shows the effect of an overshoot during heating of the block, which requires cooling back to the target temperature.

FIG. 7 depicts an artifact of some systems that is referred to as “ringing,” which involves overshoot in heating, then overshoot in cooling, and so on in progressively smaller overshoot amounts until the target temperature is reached.

In contrast to the conventional high thermal mass block, the present invention provides for unusually rapid temperature changes between the various stages of a thermocycler cycle, reducing the amount of time for each cycle, and reducing the amount of time that a reaction vial is near, but not at, each target temperature.

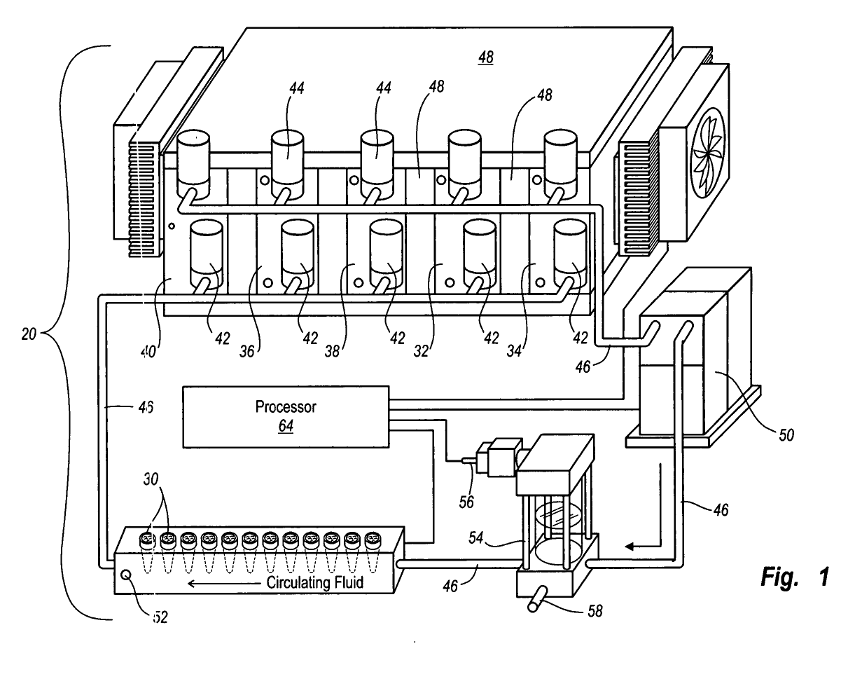

FIG. 1 depicts schematically an embodiment of a thermocycler in accordance with the present invention that is suitable for use in the practice of PCR. FIG. 1 illustrates the use of a plurality of heat exchangers that correspond to each target temperature for the various stages of a thermocycler cycle, plus an additional heat exchangers to boost cooling and heating, respectively.

Unlike a conventional thermocycler which frequently uses a high thermal mass block, the present invention increases efficiency by minimizing the thermal mass of all components other than the heat exchangers, which should have enough thermal mass to enable them to be maintained at a desired temperature during operation of the thermocycler.

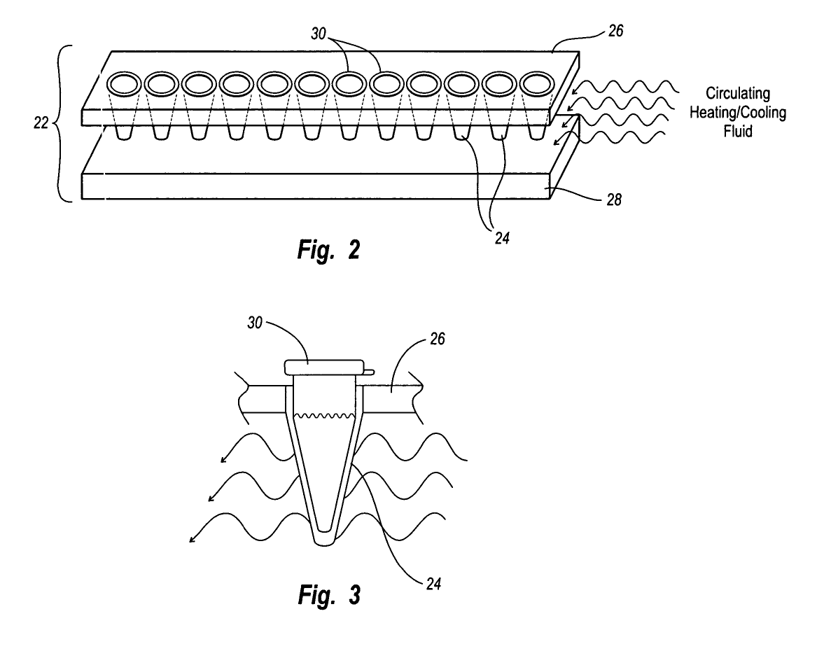

More specifically, FIG. 1 depicts a thermocycler 20 having a reaction vial holder 22, which can hold any number of reaction vials, although it is currently preferred that it be capable of holding 96 vials in the standard configuration common among conventional thermocyclers. Unlike conventional thermocyclers, however, which utilize a solid block having a high thermal mass, vial holder 22 is designed to have low thermal mass. One way of doing this is depicted schematically in FIGS. 2 and 3, which shows the use of a hollow structure rather than a solid block, with a portion of reaction vial wells 24 extending in an exposed fashion from a top vial holder element 26 into an exposed interior between the top vial holder element and bottom vial holder element 28, and between the sides of the vial holder (not shown in FIG. 2). The open interior forms a channel through which fluid is caused to flow in order to control the temperature of the reaction vial wells.

One approach for minimizing the thermal mass of vial holder 22 is to form it from a polymer or other low thermal mass material. Reaction vial wells 24 are preferably formed from a good heat conductor, such as a metal. It is preferred that vial wells 24 be formed from a thin layer of metal so that they have a small thermal mass and therefore can rapidly change from one temperature to another.

Reaction vials 30 are placed within vial wells 24 and filled with the various constituents necessary to effect PCR. The volume of the reaction vials may be conventional, or greater or less than conventional. It is preferred that the volume of reaction vials be in the range of 10 microliters to 100 microliters. Conventional reaction vials may be used, or special reaction vials may be constructed, which have a different shape or are thinner than conventional, or of a different material, such as a conductive material, or which are otherwise prepared specially for use in connection with the inventive thermocycler.

The embodiment of FIG. 1 effects temperature control through use of a plurality of heat exchangers, one for each target temperature. FIG. 1 illustrates the use of a separate heat exchanger for each of the three target temperatures for PCR, plus two additional heat exchangers to assist in rapid temperature changes.

Specifically, FIG. 1 shows the use of denaturation heat exchanger 32, which is kept at the desired denaturation temperature. Annealing heat exchanger 34 is kept at the desired temperature for the annealing step. Extension heat exchanger 36 is kept at the desired temperature for the extension step. Each of these heat exchangers are in fluid connection with reaction vial holder 22 through inflow valves 42, outflow valves 44, and tubes 46, which act as fluid flow conduits. Tubes 46 and valves 42 and 44 preferably have low thermal mass. For example, tubes 46 and valves 42 and 44 may be formed of an insulating polymer material, which not only has low thermal mass, but also assists to maintain the circulating fluid passing therethrough at a relatively constant temperature. Valves 42 and 44 may be solenoid valves, which tend to have low thermal mass.

Each heat exchanger is heated to and maintained at precisely the steady state of a desired temperature in a conventional fashion, which may be a preset temperature or may be set by the operator. It is preferred that each heat exchanger have high thermal mass in comparison to the thermal mass of the other components of the thermocycler so that it may be maintained at a desired temperature during use.

Each heat exchanger should be isolated thermally from other heat exchangers, such as through use of insulation 48.

Fluid is recirculated through the system by controlling the opening and closing of appropriate valves, and operation of pump 50, which is preferably a positive displacement micro pump having low thermal mass. A controller 64, which may be a programmable logic controller, or a dedicated or separate computer, or other structure for controlling the operation of the thermocycler, is provided for operating the various valves so as to effect the operation of the thermocycler.

A minimum of fluid is preferably used in thermocycler 20 so as to minimize the thermal mass of the thermocycler system. For PCR, it is currently preferred that Paratherm LR, manufactured by Paratherm Corporation, be used as the fluid in the system because it will not boil at the temperature range used by the system and it is odorless and will evaporate completely if spilled. Optionally, other fluids may be used, including water, oil or ethylene glycol. In one embodiment, a total volume of less than about 0.5 liters of Paratherm LR is used.

Operation of the system with a separate heat exchanger for each target temperature will provide a very useful and compact thermocycler providing improved characteristics over conventional thermocyclers. Addition of two more heat exchangers, a hot boost heat exchanger 38 and a cold boost heat exchanger 40, provides even better results. In the context of a PCR thermocycler, it is currently preferred that hot boost heat exchanger 38 be maintained at about 125 degrees C. and that cold boost neat exchanger 40 be maintained at about 4 degrees C., although one of ordinary skill will readily appreciate that alternative temperatures would also provide benefits in accordance with the teachings herein. The advantage of using a hot boost heat exchanger when raising the temperature of the circulating fluid is that it will more rapidly boost the temperature of the fluid than if the appropriate target temperature heat exchanger is used alone. For example, if the temperature is being raised to the denaturation temperature from an extension temperature, an initial period of heating is accomplished by passing the circulating fluid through hot boost heat exchanger 38, which results in very rapid heating of the contents of reaction vials 30. It has been discovered that the temperature of reaction vials 30 can be increased to within a few degrees below the temperature of denaturation heat exchanger 32 within about 3 seconds, at which point the valves to and from hot boost heat exchanger 38 are closed and those associated with denaturation heat exchanger 32 are opened so as to bring the reaction vial contents to the denaturation temperature. In accordance with one method of the invention, the reaction vial contents is brought to denaturation temperature about 2 seconds after switch over from the hot boost heat exchanger to the denaturation heat exchanger.

Cold boost heat exchanger 40 is used in a similar fashion when cooling the circulating fluid from the denaturation temperature to the annealing temperature. Again, the use of very cold circulating fluid rapidly lowers the temperature of the reaction vial contents. Again, as the temperature of the reaction vials approaches the desired annealing temperature, cold boost heat exchanger 40 is removed from the circulating fluid path, and replaced by annealing heat exchanger 34.

To assist in control of this process via. controller 64, it is preferred that reaction vial holder 22 be provided with a reaction vial simulator 52, which has the same thermal mass as a filled reaction vial 30, and which allows the controller to monitor and control the temperature of the circulating fluid through operation of the valves associated with the various heat exchangers.

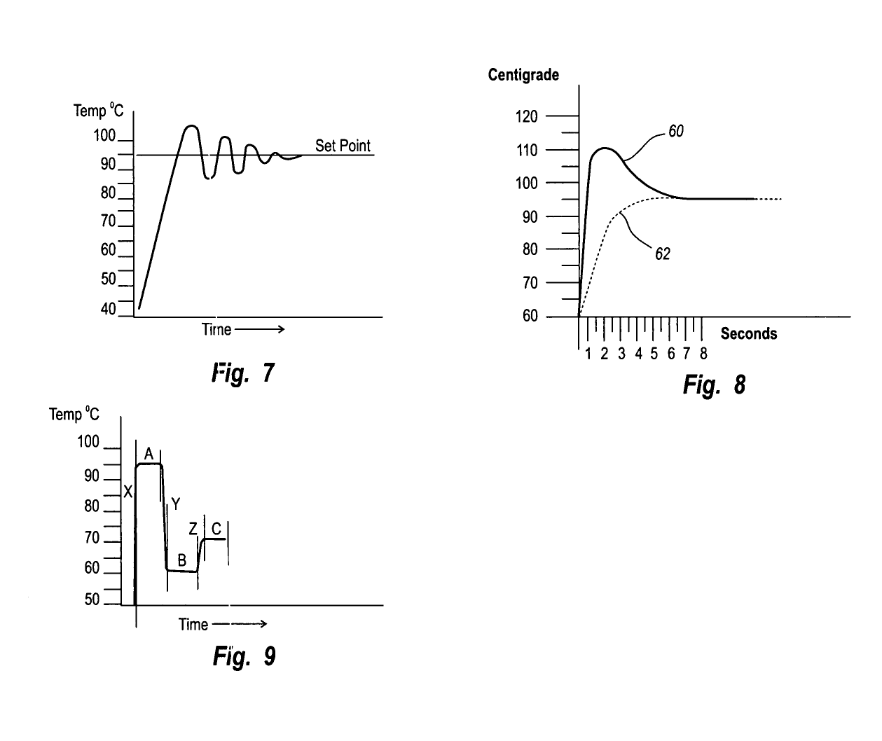

The use of a hot boost heat exchanger and a cold boost heat exchanger in combination with target temperature heat exchangers has the effect of vastly shortening the time required to move the reaction vial contents from one temperature to another. The actual temperature of the circulating fluid will rapidly change and overshoot the desired target temperature as the reaction vial contents lag behind. As the temperature of the reaction vials and the reaction vial simulator 52 approaches the target temperature and the circulating fluid is then passed through the target heat exchanger rather than the hot or cold boost heat exchanger, the temperature of the circulating fluid will then change to that of the target temperature. FIG. 8 depicts this effect. Curve 60 shows the temperature of the circulating fluid over time in one example, while curve 62 shows the rapid movement of the temperature of the reaction vial contents from 60 degrees to 95 degrees, which is the target temperature of FIG. 8. FIG. 9 shows how the temperature versus time curves for the reaction well contents approaches that of a square wave.

It is contemplated that it would be useful to provide an air and bubble catcher 54 and an air release valve 56 to assist in removing all air bubbles when setting up the thermocycler. A flow meter 58 may also be provided.

Conventional thermocyclers are bulky and heavy. The low thermal mass construction of the inventive thermocycler enables it to be very small and compact. It would be possible to take the inventive thermocycler into the field rather than requiring it to be used only within carefully controlled laboratory environments. When used in the field, the thermocycler is likely to be exposed to a wide range of conditions and ambient temperatures. This can have an effect on the operation of the thermocycler. For example, in cold environments, it is likely that there will be some cooling of heated fluid as it passes from the heat exchanger to the reaction vial holder. This cooling can be minimized through use of appropriate insulation and/or by setting the heat exchanger sufficiently above the target temperature so that the fluid will be at the target temperature when it passes through the reaction vial holder.

To account for possible variations in ambient temperature, it is preferred to run a calibration cycle prior to commencing operation of the thermocycler and to set the initial temperatures of the target heat exchangers at appropriate levels so that the reaction vial contents and reaction vial simulator will reach and be held at the target temperatures for the appropriate intervals. It is also preferred that the temperature of reaction vial simulator 52 be continually monitored during use, and that temperature adjustments to the associated target temperature heat exchanger be automatically made once per thermocycler cycle whenever simulator 52 deviates from the target temperature. By way of example, the temperature of a target temperature heat exchanger might be changed by plus or minus 0.1 degrees C. increments during each thermocycler cycle to accommodate slight shifts in ambient temperature that may occur during the period that the thermocycler is in operation.

One of ordinary skill will appreciate in view of the foregoing that the novel low thermal mass system using high thermal mass heat exchangers set to each target temperature and a circulating fluid for effecting temperature changes of reaction vials is a surprising departure from the conventional approaches and is very effective in reducing the time necessary to move reaction vial contents to the target temperatures of the thermocycler. The addition of hot boost and cold boost heat exchangers improves the system even more, providing much faster temperature changes between thermocycler stages. In view of the low thermal mass of the system, not only is it possible to obtain the benefits of faster and more controllable temperature changes, but it is possible for a thermocycler in accordance with the present invention to be much smaller than conventional thermocyclers, so much so that it is possible to provide a truly portable thermocycler which can be carried out into the field rather than requiring it to be installed in a laboratory environment.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A thermocycler, comprising:
a plurality of target temperature heat exchangers, each target temperature heat exchanger for use at a different target temperature in a thermocycler cycle;
a hot boost heat exchanger for use at a temperature above the highest target temperature;
a cold boost heat exchanger for use at a temperature below the lowest target temperature;
at least one sample holder;
fluid flow conduits communicating between the at least sample holder and the heat exchangers;
a plurality of valves for controlling fluid flow between a selected heat exchanger and the at least one sample holder;
a pump for circulating fluid through the fluid flow conduit, a selected heat exchanger, and the at least one sample holder; and
a controller that controls the flow of fluid through the various heat exchangers so as to cause the at least one sample holder to cycle through the various target temperatures.

2. The thermocycler of claim 1, wherein each heat exchanger has sufficient thermal mass to maintain its temperature during operation of the thermocycler.

3. The thermocycler of claim 1, wherein the sample holder, the fluid flow conduits, the valves and the pump have low thermal mass.

4. The thermocycler of claim 1, wherein the fluid flow conduits are formed from an insulating material.

5. The thermocycler of claim 1, wherein the volume of fluid circulated through the thermocycler is less than about 0.75 liters.

6. The thermocycler of claim 1, wherein the cold boost heat exchanger is about 4 degrees C.

7. The thermocycler of claim 1, wherein the hot boost heat exchanger is about 125 degrees C.

8. The thermocycler of claim 1, wherein the sample holder includes at least one reaction vial well, each reaction vial well being formed from a thermally conductive material, and are shaped and sized so as to receive a reaction vial.

9. The thermocycler of claim 1, wherein the sample holder includes a reaction vial simulator that reacts to changes in temperature like the at least one sample, said reaction vial simulator including a temperature measurement device that reports the temperature of the reaction vial simulator to the controller.

10. A thermocycling method, comprising the steps of:
identifying a plurality of target temperatures that a sample shall be alternately brought to;
providing a sample holder holding at least one sample to be brought to the target temperatures;
providing a fluid flow conduit communicating with the sample holder for use in setting the temperature of the sample and circulating fluid through said fluid flow conduit and said sample holder;
in the case where the target temperature is greater than the existing temperature of the sample:
heating fluid passing through said fluid flow conduit to a temperature higher than the target temperature so as to rapidly increase the temperature of the at least one sample; and
as the temperature of the at least one sample approaches the target temperature, bringing the temperature of the fluid to a temperature that will result in the at least one sample being brought to the target temperature;
in the case where the target temperature is lower than the existing temperature of the sample:
cooling fluid passing through said fluid flow, conduit to a temperature lower than the target temperature so as to rapidly decrease the temperature of the at least one sample; and
as the temperature of the at least one sample approaches the target temperature, bringing the temperature of the fluid to a temperature that will result in the at least one sample being brought to the target temperature;
maintaining the temperature of the circulating fluid at the temperature that will result in the at least one sample being at the target temperature for a desired time interval;
and repeating the steps of bringing the at least one sample to the remaining of the target temperatures for each target temperature and maintaining the at least one sample at each such target temperature for a desired time interval.

11. The method of claim 10, further comprising the step of performing a calibration step to determine what temperature that the fluid should be brought to for each target temperature in order to bring the at least one sample to the target temperatures.

12. The method of claim 10, wherein the cycle of target temperatures is repeated for a selected number of additional cycles.

13. The method of claim 12, further comprising the step of monitoring the temperature of the at least one sample at each target temperature at least once per cycle and adjusting the temperature of the circulating fluid as needed to bring the at least one sample to the respective target temperatures.

14. A thermocycler, comprising:
a plurality of target temperature heat exchangers, each target temperature heat exchanger for use at a different target temperature in a thermocycler cycle;
at least one sample holder;
fluid flow conduits communicating between the at least sample holder and the heat exchangers;
a plurality of valves for controlling fluid flow between a selected heat exchanger and the at least one sample holder;
a pump for circulating fluid through the fluid flow conduit, a selected heat exchanger, and the at least one sample holder; and
a controller that controls the flow of fluid through the various heat exchangers so as to cause the at least one sample holder to cycle through the various target temperatures.
